(19) United States
(12) Patent Application Publication
(10) Pub. No.: US 2013/0295649 A1
(43) Pub. Date: Nov. 7, 2013

(54) Photobioreactor System with High Specific Growth Rate and Low Dilution Rate

(71) Applicant: Arizona Board of Regents for and on Behalf of Arizona State University, Scottsdale, AZ (US)

(72) Inventors: Bruce Rittmann, Tempe, AZ (US); Chao Zhou, Tempe, AZ (US); Raveender Vannela, Gilbert, AZ (US)

(21) Appl. No.: 13/939,677

(22) Filed: Jul. 11, 2013

Related U.S. Application Data
(63) Continuation of application No. 13/123,057, filed on Jul. 27, 2011.
(60) Provisional application No. 61/103,474, filed on Oct. 7, 2008.

(51) Int. Cl. C12N 1/12

(52) U.S. Cl. CPC C12N 1/12
USPC 435/257.1; 435/283.1

(57) ABSTRACT

Systems and methods for growing photosynthetic cells that may be used to produce a biomass. The systems and methods recycle liquid and can produce a high cell concentration harvested biomass.

### CROSS-REFERENCE TO RELATED APPLICATIONS

This application is a continuation of co-pending U.S. patent application Ser. No. 13/123,057 filed Apr. 7, 2011, which is a national phase application under 35 U.S.C. §371 of International Application No. PCT/US2009/059651 filed Oct. 6, 2009, which claims priority to U.S. Provisional Application No. 61/103,474, filed Oct. 7, 2008. The entire contents of each of the above-referenced disclosures is specifically incorporated by reference herein without disclaimer.

### BACKGROUND OF THE INVENTION

1. Field of the Invention

Embodiments of the present invention relate generally to a system and method for growing photosynthetic cells under controlled conditions. In particular, embodiments of the present invention concern the use of photosynthetic microorganisms that can be used to produce very large amounts of biomass that can be used as a supply of renewable, carbon-neutral energy.

2. Description of Related Art

The photosynthetic microorganisms include, among others, prokaryotic cyanobacteria and eukaryotic algae. Especially when the microorganisms have high lipid content, the lipids can be extracted from the harvested biomass and converted to liquid hydrocarbon fuels, such a diesel and biodiesel. The other components in the harvested biomass can be converted to useful forms of energy, animal feed, fertilizer, and chemicals.

Photosynthetic microorganisms can be grown in closed photobioreactor systems or in open ponds. Closed photobioreactors offer a higher level of control of the microorganisms' physiology, water loss, and contamination from undesired microorganisms in the ambient environment. However, closed photobioreactors generally have higher capital costs. Therefore, one goal for photobioreactor systems is to have a high biomass yield per unit surface area and per unit volume. Since sunlight is the energy source, microbial photosynthetic systems often are located in sunny, but relatively arid environments, where a high rate of water use is not feasible. Therefore, a second goal for photobioreactor systems is to have a low water-use rate. A third goal for a photobioreactor system is that the biomass can be harvested readily and with as high a concentration as possible. The latter aspect is integral to low water use and to aid the downstream processing of the harvested biomass.

### SUMMARY

Embodiments of the present disclosure address issues related to existing systems and specifically provide for high yield rates and low water-use rates.

In order to obtain a high yield of biomass, the photosynthetic microorganisms must grow very rapidly. This is quantified by the specific growth rate, μ_{C}, which is the rate at which new biomass is synthesized (e.g., kg dry weight per day) divided by the amount of biomass in the system (e.g., kg dry weight):

μ_{C}=Q_{BH}X_{BH}/V_{CP}X_{CP} (Eqn. 1)

In Eqn. 1, Q_{BH} is the volumetric rate at which the biomass is harvested (e.g., m³/day), X_{BH} is the biomass concentration of the harvested biomass (e.g., kg dry weight/m³), V_{P} is the volume of the photobioreactor (e.g., m³), X_{CP} is the concentration of biomass in the photobioreactor (e.g., kg dry weight/m³), and μ_{C} is the specific growth rate (e.g., 1/day). A successful microbial photobioenergy system may have a specific growth rate of 1/day or larger. The rate of harvested-biomass output is given by the numerator of Eqn. 1, or Q_{BH}X_{BH}. It is desirable that this rate be high so that the maximum output is obtained for the capital costs of the photobioreactor system. Eqn. 1 can be rearranged to be:

Q_{BH}X_{BH}=μ_{C}V_{CP}X_{CP} (Eqn. 2)

From Eqn. 2, Q_{BH}X_{BH} can be maximized by making μ_{C} large, which is desirable when the objective is to maximize biomass production. Eqn. 2 also shows that the rate of biomass output is increased by a large value of X_{CP}. Thus, another objective is to have a large value of X_{CP} at the same time that μ_{C} is large.

The throughput of water can be measured with a parameter that is parallel to μ_{C}, namely the dilution rate D, which is defined as the flow-through water flow rate divided by the system volume and also has units of reciprocal time:

D=Q_{I}/V_{P} (Eqn. 3)

in which Q_{I} is the volumetric flow rate of input water to the photobioreactor system (e.g., m³/day), and D is the dilution rate (e.g., 0.1/day). It is desirable for D to be much smaller than 1/day when μ_{C} is greater than 1/day.

The harvested biomass is contained in flow rate Q_{BH} with concentration X_{BH}. It is desirable that X_{BH} have a relatively large value, because this minimizes Q_{BH} for a given rate of harvested-harvested biomass output. Minimizing Q_{BH} reduces the cost of the downstream processing of the harvested biomass. It also contributes to low water usage, since any water that is removed from the system in the harvested biomass must be added via the input flow (Q₁).

A photobioreactor operating according to these principles can therefore: (1) Allow a small D at the same time that it has a large μ_{C}; (2) Allow a high value of X_{BH} so that Q_{BH}is minimized; and (3) Allow independent control of X_{P} so that it can have a high value at the same time that μ_{C} is large.

In certain embodiments, a photobioreactor can achieve these objectives by utilizing a membrane separation device (MSD) (for example, a membrane filtration separator (MFS)). While membrane separations devices have previously been linked to other bioreactors, such devices were configured to make the specific growth rate (μ_{C}) much smaller than the dilution rate (D). In embodiments of the present disclosure, the membrane separation device is configured to achieve the diametrically opposed goal, i.e., having μ_{C} be much larger than D. Such a configuration also provides other benefits, which lead to a high production rate of biomass at the same time that the water-use rate is small. It also facilitates harvesting of the biomass and downstream processing.

Certain embodiments comprise a method of generating a biomass, where the method may include: culturing photosynthetic cells in an inner volume of one or more conduits; supplying CO₂ to the inner volume; supplying a liquid to the inner volume; supplying one or more nutrients to the inner volume; exposing the CO₂, liquid, and nutrients to light; generating a slurry containing the liquid and a generated biomass in the inner volume; removing the slurry from the inner volume; filtering the slurry to remove a harvested biomass from the slurry; and recycling the liquid to the inner volume.

In specific embodiments, the liquid may be supplied to the inner volume at a supply rate expressed in units of volume divided by units of time and the dilution rate may be expressed as the supply rate divided by the inner volume. In certain embodiments, the slurry has a slurry cell concentration expressed in units of mass per units of volume and the harvested biomass has a harvested-cell concentration expressed in units of mass per units of volume. In particular embodiments, the harvested biomass is harvested at a harvest rate expressed in units of volume per units of time and a specific growth rate is expressed as (harvest rate x harvested-cell concentration)/(slurry cell concentration x inner volume), and the dilution rate is less than the specific growth rate. In certain embodiments, the dilution rate can be less than 0.5/day, and in specific embodiments the dilution rate can be less than 0.1/day. The specific growth rate can be greater than 1.0/day in certain embodiments, and greater than 2.0/day in other embodiments.

In certain embodiments, the liquid is supplied to the inner volume at a supply rate; the liquid is recycled to the inner volume at a recycle rate; and the recycle rate is greater than the supply rate. In particular embodiments, the recycle rate can be greater than the supply rate by a factor of 5, and in certain embodiments the recycle rate can be greater than the supply rate by a factor of 10. In specific embodiments, the generated biomass and the harvested biomass may comprise cyanobacteria. In certain embodiments, the nutrient may comprise nitrogen, a component of nitrate, and/or another nitrogen compound. In specific embodiments, the nutrient may comprise phosphate and/or another phosphorous compound.

In particular embodiments, the CO₂ may be supplied by a flue gas, and in specific embodiments, the CO₂ may be supplied to the inner volume via a gas supply system comprising 0.03% to 15% CO₂. In certain embodiments, the nutrients in the inner volume can be maintained at an amount suitable for growing cyanobacteria. In particluar embodiments, the temperature in the inner volume can be maintained at a level suitable for growing cyanobacteria.

Certain embodiments may comprise a system for growing photosynthetic cells. In particular embodiments, the system can comprise: at least one conduit comprising a material that permits light to pass into an inner volume of the conduit and a CO₂ supply system configured to supply CO₂ to the inner volume during use. Certain embodiments can also comprise a liquid supply system configured to supply a liquid at a supply rate to the inner volume during use and a nutrient supply system configured to supply one or more nutrients to the inner volume during use, where the system is configured to generate within the inner volume a slurry containing the liquid and a biomass during use. Particular embodiments may also comprise a membrane filtration system configured to filter the slurry and separate a harvested biomass from a filtered liquid. Certain embodiments may also comprise a recycle system configured to recycle the filtered liquid at a recycle rate back to the inner volume. In particular embodiments of the system, the recycle rate can be greater than the supply rate. In certain embodiments, the recycle rate can be greater than the supply rate by a factor of 5, and in particular embodiments, the recycle rate can be greater than the supply rate by a factor of 10. In certain embodiments of the system, the nutrient can be a component of nitrate or another nitrogen compound and/or a component of phosphate or another phosphorous compound. In certain embodiments of the system, the biomass can comprise cyanobacteria and/or algae.

Certain embodiments may also comprise a mineral supply system configured to supply minerals to the inner volume during use. In certain embodiments, at least one conduit may be comprised of glass, clear polyvinyl chloride, or another transparent polymer. In specific embodiments, at least one conduit comprises a tube with a circular cross-section. At least one conduit may comprise a plurality of parallel tubes with a reflector between the tubes. In particular embodiments, the reflector may have a triangular cross-section.

Certain embodiments may comprise a panel configured to shield at least one conduit from sunlight. In particular embodiments, the panel can be configured to adjust positions and alter the amount of sunlight shielded from at least one conduit. Particular embodiments may comprise a sensor system configured to sense a parameter within the inner volume. In certain embodiments, the parameter may be selected from the group consisting of: temperature, pH, flow rate, CO₂ concentration and turbidity. In specific embodiments, the sensor system can be configured to provide feedback to the CO₂ supply system, the liquid supply system, and/or the nutrient supply system. In certain embodiments, the CO₂ supply system can be configured to inject flue gas into a liquid in fluid communication with the inner volume during use. Particular embodiments may comprise a pump configured to circulate the fluid within the conduit.

It is contemplated that any embodiment discussed in this specification can be implemented with respect to any method or system of the invention, and vice versa. Furthermore, systems of the invention can be used to achieve methods of the invention.

The term “conduit” or any variation thereof, when used in the claims and/or specification, includes any structure through which a fluid may be conveyed. Non-limiting examples of conduit include pipes, tubing, channels, or other enclosed structures.

The term “reservoir” or any variation thereof, when used in the claims and/or specification, includes any body structure capable of retaining fluid. Non-limiting examples of reservoirs include ponds, tanks, lakes, tubs, or other similar structures.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “inhibiting” or “reducing” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

Other objects, features, and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description. For example, certain embodiments may be configured to produce high lipid content products. Other embodiments may be configured to produce products that are not necessarily high in lipids, but have value, for example, as specialty chemicals, neutraceuticals, chemical feedstocks, or simple biomass.

### BRIEF DESCRIPTION OF THE FIGURES

FIG. 1 is a schematic view of an exemplary embodiment of photobioreactor system according to the present disclosure.

FIG. 2 is a perspective view of an exemplary embodiment of photobioreactor system according to the present disclosure.

FIG. 3 is a perspective view of an exemplary embodiment of photobioreactor system according to the present disclosure.

### DESCRIPTION OF EXEMPLARY EMBODIMENTS

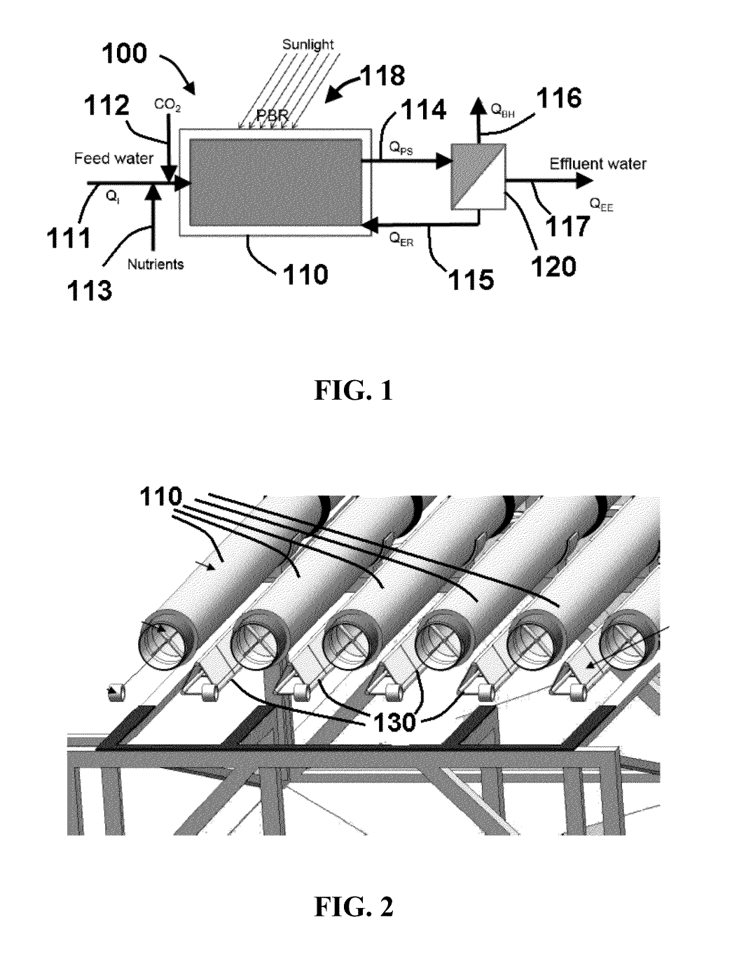

FIG. 1 is a schematic view of a photobioreactor (PBR) 100 comprising a conduit 110 and a membrane separation device (MSD) 120. PBR 100 further comprises a feed-water system 111, a CO₂ supply system 112, and a nutrient supply system configured to supply water, CO₂, and nutrients to the inner volume of conduit 110 during use. Conduit 110 is comprised from a material that permits light (such as sunlight 118) to pass into an inner volume of conduit during use.

In this embodiment, feed-water system 111 inputs water at a controlled rate consistent with the desired dilution rate D. In certain embodiments, nutrient supply system is configured to supply nitrogen and phosphorous to conduit 110. In the embodiment shown, photosynthetic cells are cultured in conduit 110 and water, CO₂, and nutrients in the inner volume are exposed to light so that a liquid slurry 114 containing biomass is formed in conduit 110.

Membrane separation device 120 is configured to separate biomass from liquid slurry 114 exiting conduit 110 (labeled as flow Q_{PS} in this embodiment). In certain embodiments, membrane separation device 120 may be comprise microfilters or ultrafilters. In specific embodiments, PBR 100 also comprises separate hydraulic connections configured to: (1) take biomass-containing slurry 114 from PBR 100 to the MSD 120; (2) separate and harvest a biomass concentrate 116 (labeled as flow Q_{BH}); (3) recycle filtered permeate 115 back to the PBR (labeled as flow Q_{ER}); (4) and to remove permeate 117 from the system (labeled as flow Q_{EE}).

As shown in this embodiment MSD 120 removes biomass from slurry 114, producing biomass concentrate 116 on the retentate side. Biomass concentrate 116 has concentration X_{BH} and is significantly higher than the biomass concentration in the slurry 114 located within conduit 110 (i.e., X_{CP}) due to the water being removed by permeation through the membrane.

Permeate flow exiting MSD 120 is divided between discharge permeate 117 (labeled as Q_{EE}) and recycle filtered permeate 115 (labeled as Q_{ER}) in order to have a low value of D, since Q_{I}=Q_{EE}+Q_{BH}, where Q_{I} is the influent flow to the photobioreactor. Even though a large amount of water is removed from the biomass by permeation, only a small portion is removed from the system in Q_{EE}. The recycling of Q_{ER} to PBR 100 allows the system to achieve a low value of D and a high value of X_{BH} during use.

The rate of biomass harvesting is Q_{BH}X_{BH} and is independent of the rate at which water enters or leaves the PBR system. By having a high value of X_{BH} in biomass concentrate 116, it is possible to have a high biomass production rate and corresponding high μ_{C} value without needing to have a high value of D.

Referring now to FIG. 2, a perspective end view of an exemplary embodiment comprises a plurality of parallel tubes or conduits 110 spaced apart. A plurality of reflectors 130 are located between adjacent conduits 110 such that each reflector 130 is parallel to each adjacent conduit 110. It is understood that reflectors 130 are optional components and that other embodiments may not comprise reflectors between the tubes. As shown in FIG. 2, each reflector 130 has a triangular cross-section. This triangular cross-section is configured so that light which would normally pass between adjacent conduits 110 is reflected back up and towards an adjacent conduit. It is understood that the spacing shown in FIG. 2 is an exemplary configuration and that other configurations are possible. For example, reflectors 130 may be positioned in a higher plane so that they are roughly in the same plane (or just slightly below) conduits 110. The exact spacing configuration can be determined based on the angle of the incoming light and the amount of light that is desired to reflect onto conduits 110.

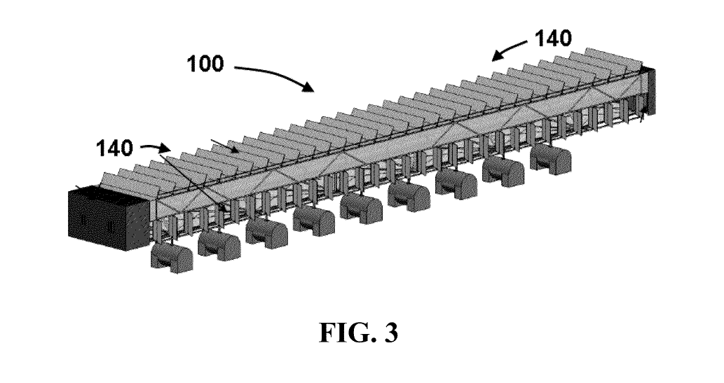

Referring now to FIG. 3, a perspective view of a PBR system 100 comprises a plurality of panels 140 configured to shield a series of conduits (not visible in FIG. 2 due to panels 140). It is understood that the panels 140 are optional components and that other embodiments may not comprise reflectors between the tubes. In certain embodiments, panels 140 can be manipulated to change positions and alter the amount of sunlight that is prevented from reaching the conduits. Panels 140 may also be used to shield conduits from other elements, including for example, rain or hail. In specific embodiments, sensors that detect one or more parameters (e.g., light intensity, temperature, etc.) may be coupled to a control system that automatically adjusts panels 140.

### Modeling Analysis

Described below is a modeling analysis that demonstrates that a PBR system such as PBR 100 can achieve the stated goals. In the model, the photosynthetic microorganisms are identified as cyanobacteria (subscript C), because they are the microorganisms that have been utilized for the experimental evaluation of the system. In other embodiments, however, algae or other photosynthetic microorganisms can be used in the system. The derivation and results apply for all photosynthetic microorganisms, not only cyanobacteria.

The first step is to define all the parameters and their symbols used in the mass-balance model:

Physical Dimensions
V_{P}=volume of the photobioreactor (m³)
V_{S}=volume of the separator (m³); (probably small compared to V_{P}).
V_{T}=total system volume (m³)=V_{S}+V_{P}

Concentrations
X_{CI}=concentration of cyanobacteria biomass in the influent (g_{C}/m³); (probably zero).
X_{CE}=concentration of cyanobacteria biomass in the effluent (permeate) (g_{C}/m³); (should be zero).
X_{CP}=concentration of cyanobacteria biomass in the photobioreactor (g_{C}/m³).
X_{CB}=concentration of cyanobacteria in the separator concentrate, which also is in the harvested biomass flow, X_{BH} (g_{C}/m³).

Volumetric Flow Rates
Q_{I}=influent flow rate (m³/day).
Q_{EE}=effluent flow rate (m³/day).
Q_{BH}=flow rate of harvested biomass from the MFS retentate (m³/day).
Q_{ER}=permeate flow rate recycled to the PBR (m³/day).
Q_{PS}=flow rate from the PBR to the MFS (m³/day).

Mass Flow Rates
M_{CI}=Q_{I}X_{CI}=mass flow rate of cyanobacteria biomass into the system (g_{C}/day); (probably zero).
M_{CE}=Q_{EE}X_{CE}=mass flow rate of cyanobacteria biomass out in the effluent (g_{C}/day); (should be zero).
M_{CH}=Q_{BH}X_{BH}=mass flow rate of cyanobacteria biomass out by harvesting (g_{C}/day).

Specific Growth Rate, Solids Retention Time, and Concentration Factor
μ_{C}=specific growth rate of cyanobacteria biomass=M_{CH}/X_{CP}V_{P} when M_{CI} and M_{CE}are zero (the usual case).
SRT_{C}=solids retention time of the cyanobacteria biomass=1/μ_{C}=X_{CP}V_{P}/M_{CH}
C.F.=biomass-concentration factor=X_{BH}/X_{CP}. C.F. depends on the operation of the MSD and properties of the biomass.

The next step is to define the mass balances that comprise the model for the cyanobacterial biomass:

Steady-State (SS) Mass Balances for the Entire System

0=−Q_{BH}X_{BH}+μ_{C}X_{CP}C_{P}=−Q_{BH}X_{BH}+X_{CP}V_{P}/SRT_{C} (Eqn. 4)

Non-Steady-State (NSS) Mass Balances for the Entire System

V_{P}dX_{CP}/dt=−Q_{BH}X_{BH}+μ_{C}X_{CP}V_{P}=−Q_{BH}X_{BH}+X_{CP}V_{P}/SRT_{C} (Eqn. 5)

Mass Balances on Biomass Around the Photobioreactor

0=−Q_{PS}X_{CP}+μ_{C}X_{CP}V_{P}(SS) (Eqn. 6)

VPdXCP/dt=−QPSXCP+μCXCPVP(NSS) (Eqn. 7)

Mass Balances on Biomass Around the Separator

0=Q_{PS}X_{CP}−Q_{BH}X_{BH}(SS) (Eqn. 8)

V_{S}dX_{BH}/dt=Q_{PS}X_{CP}−Q_{BH}X_{BH}(NSS) (Eqn. 9)

### Solving the Model

The following is a solution method for steady-state operation of an exemplary embodiment of a photobioreactor (PBR) system. It identifies what input information or choices need to be made to complete the solution:

Step 1.
Select system parameters.
Physical parameters Q_{I} and V_{P} (or V_{P} and D)
Biomass concentrations X_{CP}and X_{BH}
Specific growth rate μ_{C=l/SRT}_{C}

Step 2.
Compute M_{CH}=X_{BH}QBH=V_{P}X_{CP}μ_{C}

Step 3.
Compute Q_{B}H=M_{CH}/X_{BH}

Step 4.
Compute Q_{EE}=Q_{I}−Q_{BH}

Step 5.
Compute Q_{PS}=Q_{BH}X_{BH}/X_{CP}

Step 6.
Compute Q_{ER}=Q_{PS}−Q_{BH}−Q_{EE}

In this practice, the equations are solved for flows with specified (target) μ_{C} and D values. If any of the Q values are negative, the solution is infeasible. If all of the flows are computed as positive or zero with desirable μ_{C} and D values, then the objective is achieved.

### EXAMPLE

Presented below is an example that shows how the steps are carried out with realistic parameter values and that illustrates a feasible solution to meet the targets:
Step 1. Q_{I}=1000 m³/day, V_{P}=5,000 m³ (D=0.2 day, a realistic target value to minimize water consumption), X_{CP}=200 g/m³, X_{BH}=2,000 g/m³ (C.F. is 10 in the MSD to have a relatively high concentration of harvested biomass), μ_{C}=1/day (a realistic target value to have a high biomass production rate).
Step 2. M_{CH}=5000×200×1=10⁶ g/day.
Step 3. Q_{BH}=10⁶/2000=500 m³/day.
Step 4. Q_{EE}=1000−500=500 m³/day.
Step 5. Q_{PS}=500×2000/200=5000 m³/day.
Step 6. Q_{ER}=5000−500−500=4000 m³/day.

This example illustrates that is it possible to achieve feasible steady-state operation (all Q values are positive) with the good target parameters: μ_{C}=1/day>D=0.2 (indicating a 5-day hydraulic retention time), and X_{BH}=2,000 g/m>X_{CP}=200 g/m³.

### Systematic Analysis

The model was systematically applied to a wide range of example conditions to identify important trends and identify opportunities or problems. Some of the results are shown below.

Model Inputs values

The model was evaluated with parameters suitable for rooftop (RT) photobioreactors (PBR) coupled to a membrane-filtration system (i.e., a RT-PBR/MFS), which is being tested experimentally. The same principles and trends apply equally to larger scale systems. For modeling, the total volume of the RT-PBR/MFS system is an input parameter. For example, a volume of 2 m³=2,000 L represents an RT system. We also make C.F. an input parameter. A baseline value of biomass concentration factor (C.F.) is 20, but then the range expanded from up to 50 to explore process feasibility.

Reasonable values were selected for μ_{C} and D. A large hydraulic retention time, HRT=1/D, and a small solids retention time, SRTc (=1/μ_{C}), are desirable for this application. HRT ranged from 2 to 20 days, making D range from 0.5 to 0.05/day. SRT ranged from 0.333 to 2 days, making the μ_{C} range be 3 to 0.5/day, which are well justified by the experimental data and the literature for photosynthetic microorganisms.

### Selected Results

Table 1 summarizes six model results that illustrate the effects of systematic variation in the three key design parameters: C.F.=20 or 50; μ_{C}=2/d; D=0.2 or 0.1/d, when the biomass concentration in the photobioreactor was set at a typical value of 0.5 kg/m³. The top set is the baseline case, and [boldface] entries show changed input values from the baseline.

All six situations presented here (and many others not shown) show feasible results when μ_{C} is large (>1/day) and much larger than D (0.1 or 0.2/day), while C.F. is at least 20, making X_{BH} large (10 to 25 g/m³). Feasibility is demonstrated by having all Q values greater than or equal to 0. These results prove that the concept of having a PBR system with large specific growth rate and a low dilution rate can be achieved by the MFS configuration demonstrated here. Furthermore, the harvested-biomass concentration after the filtration can be increased by 20- to 50-fold, which means downstream processing deals with a low-volume, high-concentration slurry.

The results in Table 1 also illustrate important trends that can be used to optimize process performance. For example, for a constant value of X_{CP}, which is true for the table, the production of biomass is proportional to μ_{C}, and a large μ_{C} is desired to maximize the areal and volumetric production rates. The amount of influent (or make-up) water (Q_{I}) is minimized by having a small D, while the liquid volume for the harvested biomass (Q_{BH}) is minimized by a large C.F. The last row in the table contains all the optimized value so that productivity is at its highest value, while Q_{I} and Q_{BH} are at their smallest values.

In summary, the modeling analysis demonstrates that the novel PBR/MFS system can achieve the stated goals.

TABLE 1
Model Results with Changes in D, μ, and C.F. for a RT-scale PBR/MFS System
Baseline case

| X_{CP} | X [symbol] | X [symbol] /X_{CP} = | μ_{C} | D | SRT | [symbol] RT | |
|---|---|---|---|---|---|---|---|
| (kg/m³) | (kg/m [symbol] ) | C.F. | (1/d) | (1/d) | (d) | (d) | |
| 0.5 | 10 | 20 | 2 | 0.2 | 0.5 | 5 | |
| Q₁ | Q [symbol] | Q_{BH} | Q_{PS} | Q_{ER} | Q_{M}* | P_{V}** | P_{A}*** |
| (m³/d) | (m [symbol] /d) | (m³/d) | (m³/d) | (m [symbol] /d) | (m [symbol] /d) | (kg/(m³*d)) | (kg/(m²*d)) |
| 0.4 | 0.2 | 0.2 | 4 | 3.6 | 3.8 | 0.625 | 0.0735 |

Smaller D

| | | | | | | | |
|---|---|---|---|---|---|---|---|
| X [symbol] | X_{BH} | X_{CB}/X_{CP} = | μ_{C} | D | SRT | [symbol] RT | |
| (kg/m³) | (kg/m [symbol] ) | C.F. | (1/d) | (1/d) | (d) | (d) | |
| 0.5 | 10 | 20 | 2 | [0.1] | 0.5 | [10] | |
| Q₁ | Q [symbol] | Q_{BH} | Q_{PS} | Q_{ER} | Q_{M}* | P_{V}** | P_{A}*** |
| (m³/d) | (m [symbol] /d) | (m³/d) | (m³/d) | (m [symbol] /d) | (m³/d) | (kg/(m³*d)) | (kg/(m²*d)) |
| 0.2 | 0 | 0.2 | 4 | 3.8 | 3.8 | 0.625 | 0.0735 |

Smaller μ

[symbol]

| | | | | | | | |
|---|---|---|---|---|---|---|---|
| X [symbol] | X [symbol] | X [symbol] /X [symbol] | μ_{C} | D | SRT | [symbol] RT | |
| (kg/m [symbol] ) | (kg/m³) | [symbol] C.F. | (1/d) | (1/d) | (d) | (d) | |
| 0.5 | 10 | 20 | [1] | 0.2 | [1] | 5 | |
| Q₁ | Q [symbol] | Q [symbol] | Q_{PS} | Q_{ER} | Q_{M}* | P_{V}** | P_{A}*** |
| (m [symbol] /d) | (m [symbol] /d) | (m [symbol] /d) | (m³/d) | (m [symbol] /d) | (m³/d) | (kg/(m [symbol] *d)) | (kg/(m² [symbol] d)) |
| 0.4 | 0.3 | 0.1 | 2 | 1.6 | 1.9 | 0.313 | 0.0368 |

Smaller D and μ_{C}

| | | | | | | | |
|---|---|---|---|---|---|---|---|
| X [symbol] | X_{BH} | X_{CB}/X_{CP} | μ_{C} | D | SRT | HRT | |
| (kg/m [symbol] ) | (kg/m³) | [symbol] C.F. | ( [symbol] /d) | (1/d) | (d) | (d) | |
| 0.5 | 10 | 20 | [1] | [0.1] | [1] | [10] | |
| Q₁ | Q [symbol] | Q [symbol] | Q_{PS} | Q_{ER} | Q_{M}* | P_{V}** | P_{A}*** |
| (m³/d) | (m³/d) | (m³/d) | (m³/d) | (m [symbol] /d) | (m³/d) | (kg/(m [symbol] *d)) | (kg/(m²*d)) |
| 0.2 | 0.1 | 0.1 | 2 | 1.8 | 1.9 | 0.313 | 0.0368 |

Larger C.F.

| | | | | | | | |
|---|---|---|---|---|---|---|---|
| X_{CP} | X_{BH} | X_{CB}/X_{CP} = | μ_{C} | D | SRT | HRT | |
| (kg/m³) | (kg/m³) | C.F. | (1/d) | (1/d) | (d) | (d) | |
| 0.5 | [25] | [50] | 2 | 0.2 | 0.5 | 5 | |
| Q₁ | Q_{EE} | Q_{BH} | Q_{PS} | Q_{ER} | Q_{M}* | P_{V}** | P_{A}*** |
| (m [symbol] /d) | (m [symbol] /d) | (m³/d) | (m [symbol] /d) | (m [symbol] /d) | (m [symbol] /d) | (kg/(m³*d)) | (kg/(m²*d)) |
| 0.4 | 0.32 | 0.08 | 4 | 3.6 | 3.92 | 0.625 | 0.0735 |

Larger C.F. and smaller D

| | | | | | | | |
|---|---|---|---|---|---|---|---|
| X_{CP} | X [symbol] | X_{CB}/X_{CP} | μ_{C} | D | SRT | [symbol] RT | |
| (kg/m [symbol] ) | (kg/m³) | [symbol] C.F. | (1/d) | (1/d) | (d) | (d) | |
| 0.5 | [25] | [50] | 2 | [0.1] | 0.5 | [10] | |
| Q₁ | Q_{EE} | Q [symbol] | Q_{PS} | Q_{ER} | Q_{M}* | P_{V}** | P_{A}*** |
| (m [symbol] /d) | (m³/d) | (m [symbol] /d) | (m [symbol] /d) | (m [symbol] /d) | (m [symbol] /d) | (kg/(m [symbol] *d)) | (kg/(m²*d)) |
| 0.2 | 0.12 | 0.08 | 4 | 3.8 | 3.92 | 0.625 | 0.0735 |

Values shown in [boldface] are changes from the baseline case.
*Q_{M} is the flow rate through the membrane. It is one of the key operation parameters of the membrane and can be used to calculate the surface area needed.
v
**P_{V} is the volumetric productivity
**P_{A} is the areal productivity with horizontal cylindrical tubes of 15-cm (6″) diameter. [symbol]
indicates data missing or illegible when filed

### Experimental Manifestation

In order to experimentally demonstrate that the stated goals can be achieved with the PBR system during operation throughout the year, experiments will be performed with a roof-top photobioreactor with membrane filtration (RT-PBR/MSD) that contains approximately 2000 L of culture under controlled conditions with respect to hydraulic and solid retention times and concentrations in the PBR and the harvested biomass, as mentioned above. The PBR is comprised of transparent glass tubes with a diameter of 6 inches (15 cm) and a length of approximately 20 m. The specific growth rate (μ_{C}) of the cyanobacteria in the RT-PBR depends primarily on sunlight intensity (up to 600 W/m²), CO₂ supply, available nutrients (such as nitrate and phosphate among other), and the biomass concentration. Controlling these process parameters via the experimental design, it is expected that the specific growth rate can be controlled in the range of 1-2 per day, which corresponds to current modeling analysis. The initial experiments will be conducted to evaluate PBR performance in terms of the ability to control the specific growth rate (μ_{C}), hydraulic retention time (HRT), and biomass concentrations by coupling an MSD with the PBR.

For the MSD, a suitable filtration device from Pall Corporation has been identified that can efficiently work under these set of conditions. This system works with a cross-flow flux (CFF) of 10 Liters/minute/m², controlled permeate flux of 30-40 Liters/m²/hr, and with a pressure drop (D_{P}=P_{feed}−Pᵣₑₜₑₙₜₐₜₑ) of 2.5-3.0 psi. The above-mentioned flow rates correspond to values in the preceding table of model results and can be easily obtained using 5 m² (area) membrane.

Conditions and flow rates similar to what are shown in the table will be tested using the Pall membrane system coupled to the PBR. The Pall system is only one possibility for the membrane-separator, and it is used only to demonstrate the PBR/MSD principles.

Either continuously or periodically (semi-continuous), the biomass is pumped to the MSD unit, in this case the Pall membrane separation unit. The concentrated retentate (concentration X_{CB} in steady-state continuous operation) is then harvested as the feedstock for downstream processing (Q_{BH}).

During continuous flow mode, biomass in the PBR continuously flows to the membrane separator unit and is constantly removed from the PBR as the harvesting stream. The biomass concentration will rise gradually during the day and fall gradually at night in this case. If biomass is only harvested during daylight hours, when photosynthetic production occurs, the biomass concentration in the PBR can be kept constant. For example from Table 1, if 0.5 kg/m³ of biomass (steady-state) is in the photobioreactor growing at 2/d, a hydraulic retention time of 5 days requires that 400 L of media is replaced each day when the concentration factor is 20. The biomass that will be harvested is 20 L every 24 hours of illumination at a concentration of 5% solids, and 380 L of effluent water is removed. The total flow rate to go through 5 m² membrane is 3.8 m³/day with a permeate flux rate of 40 Liters/m²/hr (process time of 24 hrs), which is readily achievable.

The semi-continuous mode of operation will also be studied in which the biomass is cultivated in batch mode during the daytime and will be harvested after sunset. Because the biomass concentration and light intensity change with time, the growth rate is not constant. The nonsteady-state modeling under this scenario indicates that higher productivity can be achieved with the same (average) specific rates. The hydraulic loading on the membrane is higher for semi-continuous operation than with continuous operation due to the shorter period of time that is allowed to harvest. The RT-PBR/MFS provides the operational flexibility to test if we can gain the additional advantages of semi-continuous biomass harvesting.

### REFERENCES

The following references are herein incorporated by reference in their entirety.
Borowitzka, M. A. (1999). Commercial production of microalgae: ponds, tanks, tubes, and fermenters. J Biotechnol 70, 313-321.
Chisti, Y. (2007). Biodiesel from microalgae. Biotechnol Adv 25, 294-306.
Daigger, G. T, B. E. Rittmann, S. S. Adham, and G. Andreottola (2005). Are membrane bioreactors ready for widespread application? Environ. Sci. Technol. 39: 399A-406A.
Rittmann, B. E. (2008). Opportunities for renewable bioenergy using microorganisms. Biotechnol. Bioengr. 100: 203-212.
Rittmann, B. E. and P. L. McCarty (2001). Environmental Biotechnology: Principles and Applications. McGraw-Hill Book Co., New York.

What is claimed is:

1. A method of concentrating a biomass of photosynthetic cells, the method comprising:
supplying a liquid slurry containing photosynthetic cells into a membrane separation device;
contacting liquid slurry with a membrane filtration system;
generating a biomass concentrate;
generating filtered permeate;
removing the biomass concentrate from the membrane separation device; and
recycling the filtered permeate back to the source of the photosynthetic biomass.

2. The method of claim 1, the method further comprising:
generating a discharge permeate; and
removing the discharge permeate from the membrane separation device.

3. A membrane separation device (MSD) for simultaneously removing biomass and recycling the liquid medium permeate within a photobioreactor system for growing photosynthetic cells, comprising:
a housing;
a liquid slurry inflow line comprising a conduit for conducting liquid slurry, further comprising:
an inner volume;
a first end; and
a second end;
said inflow line connected at the first end to said photobioreactor and at the second and to said housing, and containing within inner volume liquid slurry;
a recycle filtered permeate outflow line comprising a conduit for conducting recycle filtered permeate, further comprising:
an inner volume;
a first end; and
a second end;
said outflow line connected at the first end to said photobioreactor and at the second end to said housing, and containing within inner volume recycle filtered permeate;
a biomass concentrate exit port in said housing, comprising a portal for conducting biomass concentrate from said housing; and
a membrane filtration system disposed within housing, configured to filter the liquid slurry and separate a harvested biomass concentrate and recycle filtered permeate.

4. The membrane separation device of claim 3, further comprising:
a discharge permeate port in said housing, comprising:
a portal for conducting discharge permeate from housing.
